(19) United States
(12) Patent Application Publication
(10) Pub. No.: US 2017/0202876 A1
(43) Pub. Date: Jul. 20, 2017

(54) STABILIZED CHLORINE DIOXIDE COMPOSITIONS AND METHODS FOR USE AS DISINFECTANTS

(71) Applicant: SKS Ag Solutions, LLC, Watertown, NY (US)

(72) Inventors: Steven PUGLIESE, Bernville, PA (US); Kevin UNDERWOOD, Canastota, NY (US); Angelo SOLURI, Watertown, NY (US); Sasha TRUDNOWSKI, Bernville, PA (US); Gregg HANSON, Huntington Valley, PA (US)

(21) Appl. No.: 15/321,436

(22) PCT Filed: Jun. 24, 2015
(86) PCT No.: PCT/US2015/037434

§ 371 Date: Dec. 22, 2016

Related U.S. Application Data
(60) Provisional application No. 62/051,615, filed on Sep. 17, 2014.
(60) Provisional application No. 62/017,020, filed on Jun. 25, 2014.

(51) Int. Cl. A61K 33/20; A61K 47/18; A61Q 17/00; A61K 8/81; A61K 8/41; A61K 47/32; A61K 8/20

(52) U.S. Cl. CPC A61K 33/20; A61K 47/32; A61K 47/186; A61K 8/20; A61K 8/8141; A61K 8/416; A61Q 17/005; A61K 2800/74; A61K 2800/52

(57) ABSTRACT

Aqueous anti-microbial and cosmetic compositions are provided that comprise chlorine dioxide, wherein the chlorine dioxide is stabilized by two quaternary ammonium cations (“quats”). These quat-stabilized chlorine dioxide compositions can also comprise a dye, a surfactant or surfactant blend, a thickener, a foam booster and/or a fragrance. The quat-stabilized chlorine dioxide compositions can be used for sanitizing and conditioning the skin of humans and animals.

### CROSS-REFERENCE TO RELATED APPLICATIONS

This application claims priority to and the benefit of co-pending U.S. provisional patent application Ser. No. 62/017,020 entitled Stabilized Chlorine Dioxide Compositions and Methods for Use as Disinfectants, filed Jun 25, 2014, and U.S. provisional patent application Ser. No. 62/051,615, entitled Stabilized Chlorine Dioxide Compositions and Methods for Use as Skin Cleansers, filed Sep. 17, 2014, both of which are incorporated herein by reference in their entireties.

### 1. TECHNICAL FIELD

The present invention relates to stabilized chlorine dioxide compositions. The invention also relates to methods for using stabilized chlorine dioxide compositions as disinfectants and germicides.

### 2. BACKGROUND

Chlorine Dioxide (CIO₂)

Chlorine dioxide (CIO₂) is well known in the art as an oxidizing and sterilizing agent and disinfectant. Chlorine dioxide has EPA approval for many applications, including for use as an anti-inflammatory, bactericidal, fungicidal, and virucidal agent, as well as a deodorizer and an anti-spoilage agent. Effective in highly contaminated systems, CIO₂ is unmatched as a disinfectant in systems contaminated with hydrocarbon because it reacts very slowly, if at all, with many organics.

Chlorine dioxide acts by oxygenating the substances with which it reacts, rather than by chlorinating them. Chlorine dioxide reacts more weakly and more selectively with the substances with which it reacts than do chlorine gas or hypochlorite compositions. This reaction of chlorine dioxide with other substances generates oxygenated byproducts, which are less hazardous and toxic than chlorinated byproducts such as dioxins, trihalomethanes (THMs), and haloacetic acids (HAAs). Because of chlorine dioxide's low reactivity, its antimicrobial activity persists longer after application than that of chlorine gas or hypochlorite compositions. When the cell membranes of microorganisms are oxidized by chlorine dioxide, rapid death ensues.

In a number of applications, CIO₂ has been found to be very effective at removal, prevention and control of biofilm, especially anaerobic biofilm. CIO₂ is polar, with a polarity that falls somewhere between that of hexane and that of water. Consequently, CIO₂ rapidly distributes into the organic phase, unlike ionic or charged species (such as hypochlorite ion), or polar molecules, (such as water).

CIO₂ has been found to be effective at eliminating bacteria at applied dosages that are well below the demand. CIO₂ allows for slow bacterial recovery. Many non-oxidizing biocides used for bacterial control are substances which may only injure bacteria, so that they can recover. CIO₂ provides a complete kill. Bacteria have not been found which are able to develop resistance to inactivation by CIO₂. C₂, moreover, shows disinfection success effective over a wide range of pH. CIO₂ is effective over a range of pH from about 4 to about 10.

Chlorine dioxide has been recognized for its powerful disinfecting properties since the early 1900s. In fact, municipalities use it to purify drinking water. Environmentalists now recommend its use in eco-friendly paper production.

CIO₂ is considered environmentally friendly. CIO₂, unlike chlorine, does not react with organics to produce carcinogenic trihalomethanes (THMs)

This fact, coupled with the fact that, ultimately, CIO₂ breaks down to chloride ion, makes it the only true “green” biocide in use today.

Chlorine dioxide, however, is an unstable gas that is typically generated on demand or as needed. Chlorine dioxide gas is hazardous to handle and has an unattractive odor, both as a gas and in aqueous solution. Thus although chlorine dioxide is effective for use as an anti-microbial agent, its drawbacks are instability, unattractive odor and the safety requirement that it be generated on demand.

Mastitis

Mastitis, a mammary gland infection affecting udder tissue, is one of the most common diseases of dairy cattle, as well as very costly. In mastitis infections, mammary gland tissue is invaded by bacteria and can be damaged by toxins released by the bacteria. Mastitis infections can cause swelling, pain and even death in infected cows, as well as produce abnormalities in milk composition. Mastitis bacterial infections can be spread by contact with contaminated human hands, milking machines, or other contaminated materials. Thus germicidal and disinfectant teat dips, sprays, or foams are popular for proper milking hygiene to prevent the spread of mastitis. Foam, in particular, is advantageous because it is more economical and efficient, serving as a better form of disinfectant that is more easily dispersed for effective cleaning.

Furthermore, the production of chlorine dioxide is known in the art to entail the use of a two-part system to generate fresh chlorine dioxide. It is difficult to control the quantity and/or the release of the chlorine dioxide that is generated using this system.

Citation or identification of any reference in Section 2, or in any other section of this application, shall not be considered an admission that such reference is available as prior art to the present invention.

### 3. SUMMARY

An aqueous composition is provided comprising quat-stabilized chlorine dioxide and water.

In one embodiment, the composition is used as a teat dip for dairy mammals, e.g., cow, goat, sheep, buffalo, camel, alpaca, or yak. In one embodiment, the composition for use as a teat dip comprises about 1-10,000 ppm of quat-stabilized chlorine dioxide. In other embodiments, the composition comprises about 10-5,000, 100-200, 200-300, 300-400, 400-500, 500-1000, 1000-2000, 2000-3000, 3000-4000, 4000-5000 or 5000-6000, 6000-7000, 7000-8000, 8000-9000 or 9000-10,000 ppm of quat-stabilized chlorine dioxide. In a specific embodiment, the composition comprises 300 ppm of quat-stabilized chlorine dioxide.

In another embodiment, the composition is used as a dry cow or dairy mammal dip for dairy mammals, e.g., cow, goat, sheep, buffalo, camel, alpaca, or yak. In one embodiment, the composition for use as a dry cow or dairy mammal dip comprises about 1-10,000 ppm of quat-stabilized chlorine dioxide. In other embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 300 ppm of quat-stabilized chlorine dioxide.

In another embodiment, the composition is for use as a foot and/or hoof spray or dip for mammals with hooves such as cow, pig, horse, donkey, mule, goat, sheep, buffalo, camel, alpaca, or yak. In one embodiment, the composition for use as a foot and/or hoop spray or dip comprises about 1-10,000 ppm of quat-stabilized chlorine dioxide. In other embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 3000 ppm of quat-stabilized chlorine dioxide.

In another embodiment, the composition is for use as a disinfectant applied to navels of calves or other newborn or young offspring of domestic mammals (e.g., pig, horse, donkey, mule, goat, sheep, buffalo, camel, alpaca, or yak). In one embodiment, the composition for use as a as a disinfectant applied to navels comprises about 1-10,000 ppm of quat-stabilized chlorine dioxide. In other embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 2000 ppm of quat-stabilized chlorine dioxide.

In another embodiment, the composition is for use as a skin sanitizer or cleanser that can be used, e.g., on the skin of mammals, including but not limited to humans, dairy mammals and other domestic animals (e.g., cats, dogs, etc.). In certain embodiments, the skin sanitizer can be used, e.g., as a hand sanitizer, a facial or body wash, a surgical scrub, acne cleanser or in a sanitizing wipe. In various embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 300 ppm of quat-stabilized chlorine dioxide.

In another embodiment, the composition comprises a dye, a surfactant or surfactant blend, a thickener, a foam booster and/or a fragrance.

In another embodiment of the composition, the surfactant or surfactant blend is a film-forming surfactant or surfactant blend.

In another embodiment of the composition, the surfactant or surfactant blend is also the thickener.

In another embodiment of the composition, the surfactant or surfactant blend comprises ECOSURF™ EH-9, sodium lauryl sulfate (SLS), sodium lauryl ether sulfate (SLES), or cocamidobetaine.

In another embodiment of the composition, the quat-stabilized chlorine dioxide is at a concentration of about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm in a surfactant base (e.g., a commercial preparation such as Vital Oxide, VitalOxide Co., 3755 Fiscal Ct. Suite B, West Palm Beach Fla. 33404).

In another embodiment of the composition, the composition comprises acrylates copolymer. In another embodiment, the thickener is an acrylates copolymer.

In another embodiment of the composition, the acrylates copolymer is CARBOPOL® Aqua SF-1 or SL Polymer.

In another embodiment, the thickener is a lightly cross-linked acrylate copolymer such as CARBOPOL® Aqua SF-1 or SL Polymer (Lubrizol, Wickliffe, Ohio) or cross-linked polyacrylic acid polymer such as CARBOPOL® ULTREZ-10 (Lubrizol, Wickliffe, Ohio).

In another embodiment of the composition, the composition comprises a dispersant. Dispersants are well known in the art, e.g., SPECTRAGUARD™ antiscalant/dispersant (a proprietary blend of caprylyl glycol, hexylene glycol and methylisothiazolinone; Professional Water Technologies, Vista, Calif.), 0.80%.

In another embodiment of the composition, the composition comprises a polydimethylcyclosiloxane (e.g., a polydimethylcyclosiloxane composed mainly of decamethylcyclopentasiloxane) such as Dow Corning® ST -Cyclomethicone 5- NF.

A method is provided for treating a teat of a mammal comprising applying to the teat the aqueous quat-stabilized chlorine dioxide composition.

In one embodiment of the method, the treating is disinfecting, sanitizing or conditioning.

In another embodiment of the method, the mammal is a dairy mammal, e.g., cow, goat, sheep, buffalo, camel, alpaca, or yak.

In another embodiment of the method, the mammal is a cow, pig, horse, donkey, goat, sheep, buffalo, camel, alpaca, or yak.

In another embodiment of the method, the mammal is dry or non-lactating.

A method is provided for treating skin comprising applying to the skin the aqueous quat-stabilized chlorine dioxide composition.

In various embodiments of the method, the treating is disinfecting, sanitizing (e.g., as a hand sanitizer), cleaning, conditioning, exfoliating, acne treatment, or skin fungus treatment.

For example, the aqueous quat-stabilized chlorine dioxide composition can be combined with exfoliating agents such as PRP® FG beeswax, beads, abrasive particles, or other abrasives or exfoliants known in the art.

In another embodiment of the method, the composition comprises a dye, a surfactant or surfactant blend, a thickener, a foam booster and/or a fragrance.

In another embodiment of the method, the surfactant or surfactant blend is a film-forming surfactant or surfactant blend.

In another embodiment of the method, the surfactant or surfactant blend is also the thickener.

In another embodiment of the method, the surfactant or surfactant blend comprises ECOSURF™ EH-9, sodium lauryl sulfate (SLS), sodium lauryl ether sulfate (SLES), or cocamidobetaine.

In another embodiment of the composition, the composition comprises acrylates copolymer. In another embodiment, the thickener is an acrylates copolymer.

In another embodiment of the method, the acrylates copolymer is CARBOPOL® Aqua SF-1 or SL Polymer.

A method is provided for treating mastitis in a mammal comprising applying to a teat of the mammal the aqueous quat-stabilized chlorine dioxide composition.

In one embodiment of the method, the treating is disinfecting, sanitizing or conditioning.

In another embodiment of the method, the mammal is a dairy mammal e.g., cow, goat, sheep, buffalo, camel, alpaca, or yak.

In another embodiment of the method, the mammal is a cow, pig, horse, donkey, goat, sheep, buffalo, camel, alpaca, or yak.

In another embodiment of the method, the mammal is dry or non-lactating.

In another embodiment of the method, the composition comprises a dye, a surfactant or surfactant blend, a thickener, a foam booster and/or a fragrance.

In another embodiment of the method, the surfactant or surfactant blend is a film-forming surfactant or surfactant blend.

In another embodiment of the method, the surfactant or surfactant blend is also the thickener.

In another embodiment of the method, the surfactant or surfactant blend comprises ECOSURF™ EH-9, sodium lauryl sulfate (SLS), sodium lauryl ether sulfate (SLES), or cocamidobetaine.

In another embodiment of the composition, the composition comprises acrylates copolymer. In another embodiment, the thickener is an acrylates copolymer.

In another embodiment of the method, the acrylates copolymer is CARBOPOL® Aqua SF-1 Polymer.

A method is provided for treating a mammalian navel comprising applying to the mammalian navel the aqueous quat-stabilized chlorine dioxide composition.

In one embodiment of the method, the treating is disinfecting, sanitizing or conditioning.

In another embodiment of the method, the mammal is a dairy mammal, e.g., cow, goat, sheep, buffalo, camel, alpaca, or yak.

In another embodiment of the method, the mammal is a cow, pig, horse, donkey, mule, goat, sheep, buffalo, camel, alpaca, or yak.

In another embodiment, the mammal is a newborn or young mammal.

In another embodiment of the method, the composition comprises a dye, a surfactant or surfactant blend, a thickener, a foam booster and/or a fragrance.

In another embodiment of the method, the surfactant or surfactant blend is a film-forming surfactant or surfactant blend.

In another embodiment of the method, the surfactant or surfactant blend is also the thickener.

In another embodiment of the method, the surfactant or surfactant blend comprises ECOSURF™ EH-9, sodium lauryl sulfate (SLS), sodium lauryl ether sulfate (SLES), or cocamidobetaine.

In another embodiment of the composition, the composition comprises acrylates copolymer. In another embodiment, the thickener is an acrylates copolymer.

In another embodiment of the method, the acrylates copolymer is CARBOPOL® Aqua SF-1 Polymer.

A method is provided for treating a hoof of a mammal comprising applying to the hoof the aqueous quat-stabilized chlorine dioxide composition.

In one embodiment of the method, the treating is disinfecting, sanitizing or conditioning.

In another embodiment of the method, the mammal is a dairy mammal, e.g., cow, goat, sheep, buffalo, camel, alpaca, or yak.

In another embodiment of the method, the mammal is a mammal with hooves, such as a cow, pig, horse, donkey, mule, goat, sheep, buffalo, camel, alpaca, or yak.

In another embodiment of the method, the composition comprises a dye, a surfactant or surfactant blend, a thickener, a foam booster and/or a fragrance.

In another embodiment of the method, the e surfactant or surfactant blend is a film-forming surfactant or surfactant blend.

In another embodiment of the method, the surfactant or surfactant blend is also the thickener.

In another embodiment of the method, the surfactant or surfactant blend comprises ECOSURF™ EH-9, sodium lauryl sulfate (SLS), sodium lauryl ether sulfate (SLES), or cocamidobetaine.

In another embodiment of the composition, the composition comprises acrylates copolymer. In another embodiment, the thickener is an acrylates copolymer.

In another embodiment of the method, the acrylates copolymer is CARBOPOL® Aqua SF-1 Polymer.

### 4. BRIEF DESCRIPTION OF THE DRAWINGS

The present invention is described herein with reference to the accompanying drawing(s), in which similar reference characters denote similar elements throughout the several views. It is to be understood that in some instances, various aspects of the invention may be shown exaggerated, enlarged, exploded, or incomplete to facilitate an understanding of the invention.

FIG. 1. Photograph of an embodiment of an aqueous quat-stabilized chlorine dioxide foaming teat dip.

FIG. 2. Photographs of test plates after germiculture. See Section 6.3, Example 3 for more details.

### 5. DETAILED DESCRIPTION

For clarity of disclosure, and not by way of limitation, the detailed description of the invention is divided into the subsections set forth below.

Stabilized Chlorine Dioxide Compositions

Aqueous anti-microbial and cosmetic compositions are provided that comprise chlorine dioxide and water, wherein the chlorine dioxide is stabilized by two quaternary ammonium cations (“quats”). These compositions, referred to herein as “quat-stabilized” chlorine dioxide compositions, can also comprise a cosmetic base, a thickener, a dye, a surfactant, a thickener, a foam booster and/or a fragrance. The compositions quat-stabilized chlorine dioxide, when combined with a film-forming and/or thickening surfactant or surfactant blend, can also serve as an emollient system. The quat-stabilized chlorine dioxide compositions can be used for disinfecting, cleaning and/or conditioning the skin of mammals including humans and domestic animals (e.g., on teat skin, navels and hooves of mammals). The quat-stabilized chlorine dioxide compositions are safe on skin, conditioning to the skin and has longer-term release of anti-microbial action than currently available chlorine dioxide-based products.

Chlorine Dioxide

Chlorine dioxide (Chemical Abstracts Service Registry Number (CASRN) 10049-04-4) is listed as a Manufacturing Use Product (MUP) by the EPA. Methods for preparing chlorine dioxide are well known in the art. In the laboratory, CIO₂ is prepared by oxidation of sodium chlorite:

2 NaCIO₂+Cl₂→2 CIO₂+2 NaCl

Methods for making chlorine dioxide (or obtaining chlorine dioxide commercially) are well known in the art. The chlorine dioxide of the compositions disclosed herein is stabilized with quaternary ammonium cations (“quats”; see below).

Quat-stabilized chlorine dioxide can be obtained from commercially, e.g., Vital Oxide (VitalOxide Co., West Palm Beach Fla.). In another embodiment, the quat-stabilized chlorine dioxide is comprised in a proprietary blend, Dry Cow Resin (Advanced Skin Technologies Inc., P.O. Box 579, 7143 Bernville Rd., Bernville, Pa. 19506).

Quaternary Ammonium Cations (“Quats”)

Quaternary ammonium cations are positively charged polyatomic ions of the structure NR₄⁺:

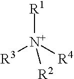

where R¹, R², R³, or R⁴ are the same or different aryl or alkyl group. In certain embodiment, R¹, R², R³, or R⁴ can be connected.

Synthesis of quaternary ammonium compounds is well known in the art. They can be prepared by quaternization, i.e., alkylation of tertiary amines.

Cosmetic Base

The compositions provided herein can be used as teat dips, dry cow (or dairy mammal) dips, navel dips or hoof sprays, skin sanitizers, cleansers (including exfoliating cleansers), and disinfectants. The composition can also comprise a cosmetic base that can, in certain embodiments, serve as a delivery system for delivering the quat-stabilized chlorine dioxide to the human or animal to be treated with the sanitizer, cleanser, disinfectant, dip or spray. The cosmetic base can be used to suspend, stabilize, and thicken the composition.

In one embodiment, the cosmetic base is an acrylates copolymer (also referred to herein as acrylate polymer or acrylate copolymer), which is a lightly cross-linked acrylates copolymer. Acrylates copolymer refers to copolymers of two or more monomers consisting of acrylic acid, methacrylic acid or one of their simple esters. The acrylates copolymer is a liquid, acrylic rheology modifier that can be used to suspend, stabilize, thicken, and enhance the appearance of surfactant-based and soap-based cleaning systems, and is a well-known ingredient of cosmetics.

Thus in one embodiment of the composition, the composition comprises acrylates copolymer. In another embodiment, the thickener is an acrylates copolymer.

In an embodiment, the acrylates copolymer is CARBOPOL® Aqua SF-1 or SL Polymer (Lubrizol, Wickliffe, Ohio).

The cosmetic base can be employed as a film former and/or thickener in various embodiments. In one embodiment, the cosmetic base, as mentioned above, comprises acrylates copolymer such as CARBOPOL® Aqua SF-1 Polymer. A cosmetic base such as an acrylates copolymer can be used in the compositions provided herein to uniformly coat or distribute chlorine dioxide anti-bacterial activity onto the surface on which it is coated. The cosmetic base can be used to increase and prolong the germicidal and skin conditioning activity of the quat-stabilized chlorine dioxide, and can be used to further increase the stability of the aqueous composition comprising quat-stabilized chlorine dioxide and water.

Dye

In one embodiment, the quat-stabilized chlorine dioxide composition comprises a dye. Unlike chlorine dioxide, which is known in the art to be incompatible with most staining (as opposed to pigment) dyes, the compositions provided herein comprising quat-stabilized chlorine dioxide can be used with most non-toxic dyes for histological staining. Routine methods can be used to determine whether a non-toxic dye is suitable for use with quat-stabilized chlorine dioxide.

The dye can be used to mark the surface being treated. In one embodiment, the quat-stabilized chlorine dioxide composition is used as a teat dip for dairy cows and the dye is used to mark and identify animals treated with the quat-stabilized chlorine dioxide composition. In another embodiment, the dye is used in a navel dip or spray to mark the navels of calves. It is well known in the art that the survival rate of calves is improved if their navels are dipped.

The dye can be used to enhance the appearance of the composition or to mark a skin surface being treated.

The dye can be a stable dye such as methylene blue, blue #1 or pigment RED ST-2; however, any stable dye known in the art to be suitable for coloring cosmetic formulations and/or for marking animal skin, such as calf navels or cow teats, can be used.

The dye can also be used to mark and distinguish between various embodiments and formulations, e.g., various embodiments of sanitizers that are color-coded, or hoof spray that is color-coded a different color from a navel spray or a teat dip.

Surfactants and Foam Boosters

In various embodiments, the quat-stabilized chlorine dioxide composition can be a film-forming aqueous composition or foam.

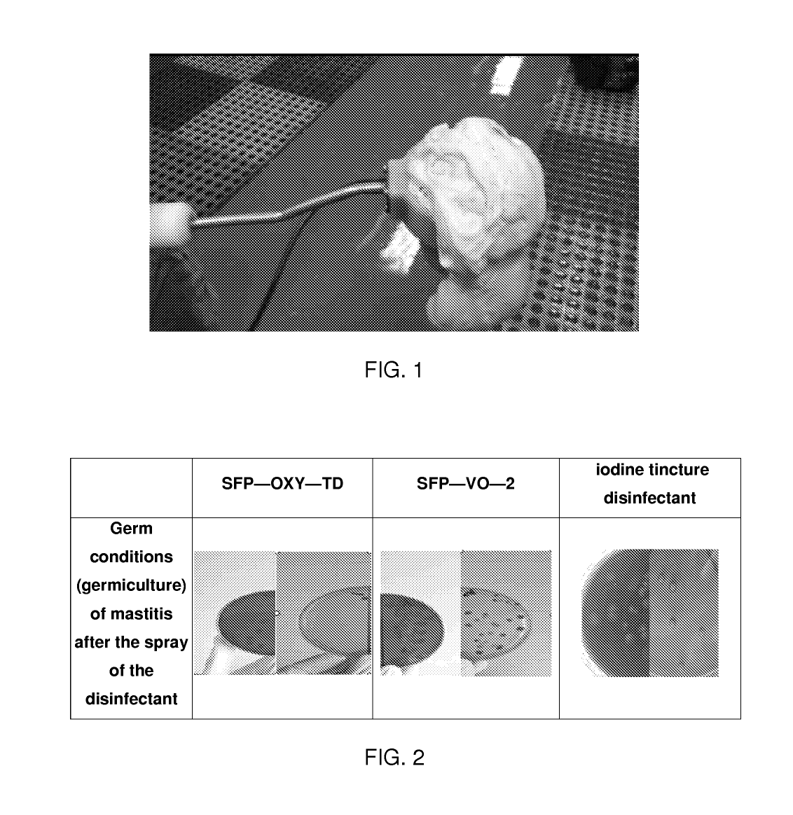

In various embodiments, the quat-stabilized chlorine dioxide composition comprises a surfactant or a plurality of surfactants, which can, in certain embodiments, also serve as foam boosters or film formers. Surfactants are compounds well known in the art that lower the surface tension (or interfacial tension) between two liquids or between a liquid and a solid. The surfactant may act as a foaming agent or foam booster (FIG. 1), as well as a film former, detergent, wetting agent, emulsifier, or dispersant.

In an embodiment, the surfactant is a nonionic surfactant. In another embodiment, the nonionic surfactant is a long chain alcohol known such as fatty alcohols, cetyl alcohol, stearyl alcohol, and cetostearyl alcohol, and oleyl alcohol. In another embodiment, the nonionic surfactant can be selected from the group consisting of:
Cetomacrogol 1000
Cetostearyl alcohol
Cetyl alcohol
Polyoxyethylene glycol alkyl ethers (Brij): CH₃—(CH₂)₁₀₋₁₆—(O—C₂H₄)₁₋₂₅—OH:
Octaethylene glycol monododecyl ether
Pentaethylene glycol monododecyl ether
Polyoxypropylene glycol alkyl ethers: CH₃—(CH₂)₁₀₋₁₆—(O—C₃H₆)₁₋₂₅—OH
Glucoside alkyl ethers: CH₃—(CH₂)₁₀₋₁₆—(O-Glucoside)₁₋₃—OH
Decyl glucoside
Lauryl glucoside
Octyl glucoside
Polyoxyethylene glycol octylphenol ethers: C₈H₁₇—(C₆H₄)—(O—C₂H₄)₁₋₂₅—OH:
Triton X-100
Polyoxyethylene glycol alkylphenol ethers: C₉H₁₉—(C₆H₄)—(O—C₂H₄)₁₋₂₅—OH:
Glycerol alkyl esters
Glyceryl laurate
Polyoxyethylene glycol sorbitan alkyl esters
Sorbitan alkyl esters
Cocamide MEA
Cocamide DEA
Dodecyldimethylamine oxide
Block copolymers of polyethylene glycol and polypropylene glycol
Polyethoxylated tallow amine (POEA)
IGEPAL CA-630
lsoceteth-20
Monolaurin
Nonidet P-40
NP-40
Octaethylene glycol monododecyl ether
N-Octyl beta-D-thioglucopyranoside
Octyl glucoside
Oleyl alcohol
Pentaethylene glycol monododecyl ether
Poloxamers
Poloxamer 407
Polyglycerol polyricinoleate
Polysorbate
Polysorbate 20
Polysorbate 80
Sorbitan monostearate
Sorbitan tristearate
Stearyl alcohol
Triton X-100
Tween 80

In one embodiment, the surfactant is the nonionic surfactant ECOSURF™ (Dow Chemical Company, Midland, Mich.). In another embodiment, the surfactant is cocamidobetaine, sodium lauryl sulfate (SLS), or sodium lauryl ether sulfate (SLES). Combinations of surfactants (“surfactant blends”), such as combinations of ECOSURF™, cocamidobetaine, sodium lauryl sulfate (SLS), or sodium lauryl ether sulfate (SLES) can also be used. Use of surfactants in skin treatment formulations (e.g., lotions, cleansers, sanitizers, etc.) is well known in the art.

In certain embodiments, such as teat dip, a foam booster is used. The foam booster can be used to intensify the germicidal and anti-microbial activities of the quat-stabilized chlorine dioxide compositions by producing a clinging foam.

In other embodiments, a foam booster can be used to intensify, by promoting foaming, the cleaning activity of the dip (FIG. 1).

For example, in a pre-milking teat dip the foam booster can be used to intensify the cleaning activity of the dip.

In other embodiments, (e.g., skin cleansers, post-milking teat dip), the foam booster can be used to promote the formation of a clinging foam for effective application of the cleanser or dip.

Embodiments in foam form have the economic advantage of allowing more coverage using less of the quat-stabilized chlorine dioxide composition.

Methods for Making and Using Quat-Stabilized Chlorine Dioxide Compositions

The quat-stabilized chlorine dioxide compositions are unexpectedly useful for disinfecting, sanitizing, cleaning and conditioning human skin and animal skin (e.g., teat skin), navels and hooves). The compositions are surprisingly gentle, non-drying (do not extract moisture from the skin), and are skin conditioning.

The quat-stabilized chlorine dioxide composition can take any suitable form known in the art, such as an aqueous solution, foam or a film-forming solution. When combined with film formers or in the form of foams, they maintain their disinfecting, sanitizing, cleaning and conditioning properties and stay in place where applied on the skin, for longer periods than do aqueous solutions without film formers or not in the form of foams.

The quat-stabilized chlorine dioxide compositions can therefore be used successfully in a variety of human cleansing and animal husbandry applications.

It will be clear to the skilled practitioner, however, that the quat-stabilized chlorine dioxide compositions can be used in disinfecting, sanitizing and skin conditioning applications such as veterinary applications, including for use with companion animals, zoo animals, exotic animals or wild animals.

In one embodiment, a quat-stabilized chlorine dioxide composition is provided that can be used at concentrations of:

About 1-10,000 ppm of quat-stabilized chlorine dioxide, for use as a skin sanitizer, cleanser or disinfectant or in a skin sanitizing wipe. Preparation of skin wipes comprising sanitizing ingredients is well known in the art. In other embodiments, the composition comprises about 10-5,000, 100-200, 200-300, 300-400, 400-500, 500-1000, 1000-2000, 2000-3000, 3000-4000, 4000-5000 or 5000-6000, 6000-7000, 7000-8000, 8000-9000 or 9000-10,000 ppm of quat-stabilized chlorine dioxide. In a specific embodiment, the composition comprises 300 ppm of quat-stabilized chlorine dioxide.

About 1-10,000 ppm of quat-stabilized chlorine dioxide, for use as a teat dip for dairy mammals, e.g., cow, goat, sheep, buffalo, camel, alpaca, or yak. In other embodiments, the composition comprises about 10-5,000, 100-200, 200-300, 300-400, 400-500, 500-1000, 1000-2000, 2000-3000, 3000-4000, 4000-5000 or 5000-6000, 6000-7000, 7000-8000, 8000-9000 or 9000-10,000 ppm of quat-stabilized chlorine dioxide. In a specific embodiment, the composition comprises 300 ppm of quat-stabilized chlorine dioxide.

About 1-10,000 ppm of quat-stabilized chlorine dioxide, for use as a dry cow (or dairy mammal) dip for dairy mammals, e.g., cow, goat, sheep, buffalo, camel, alpaca, or yak. In other embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 300 ppm of quat-stabilized chlorine dioxide.

About 1-10,000 ppm of quat-stabilized chlorine dioxide for use as a foot and/or hoof spray or dip for mammals with hooves such as cow, pig, horse, donkey, mule, goat, sheep, buffalo, camel, alpaca, or yak. In other embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 3000 ppm of quat-stabilized chlorine dioxide.

About 1-10,000 ppm of quat-stabilized chlorine dioxide for use as a disinfectant applied to navels of calves or other newborn or young offspring of domestic mammals (e.g., pig, horse, donkey, mule, goat, sheep, buffalo, camel, alpaca, or yak). In other embodiments, the composition comprises about 1-10, 10-100, 100-200, 200-300, 300-400, 400-500, 500-600, 600-700, 700-800, 800-900, 900-1000,1000-2000, 2000-3000, 3000-4000, 4000-5000, 5000-6000, 6000-7000, 7000-8000, 8000-9000 or 9000-10,000 ppm of quat-stabilized chlorine dioxide. In a specific embodiment, the composition comprises 2000 ppm of quat-stabilized chlorine dioxide.

In other embodiments, the quat-stabilized chlorine dioxide compositions can be used to disinfect, sanitize and/or condition the teats, feet, hooves and navels of domestic animals in addition to cows, such as pig, horse, donkey, mule, goat, sheep, buffalo, camel, alpaca, or yak.

In another embodiment, the quat-stabilized chlorine dioxide compositions can be used as a dry (non-lactating) mammal teat dip to inhibit or prevent mastitis in mammals such as dairy mammals, e.g., cow, goat, sheep, buffalo, camel, alpaca, or yak.

In another embodiment, the quat-stabilized chlorine dioxide composition is an aqueous concentrated composition having 10,000 ppm quat-stabilized chlorine dioxide, which can be diluted to various dilution ranges as desired for a desired use as a germicide or disinfectant, as disclosed herein.

The quat-stabilized chlorine dioxide composition kills with a gentle, lasting kill that is surprisingly skin conditioning. In mammals such as dairy cattle, for example, teats treated with the quat-stabilized chlorine dioxide composition were softer, had better color and generally appeared to be healthier, more supple and in better condition (“hand”) than teats treated with a traditional (non-quat stabilized) chlorine dioxide composition (standard 2-part mix formulation).

The quat-stabilized chlorine dioxide composition can be applied to mammalian skin repeatedly and/or continuously (e.g., 1 minute of immersion or exposure) with no undesirable effects. The surprisingly effective skin conditioning properties of quat-stabilized chlorine dioxide are attributed to the quat-stabilization of the chlorine dioxide.

The quat-stabilized chlorine dioxide composition remains consistent in extreme variations of temperature from cold to hot, whereas conventional hydrogen peroxide-based and non-stabilized chlorine dioxide-based products usually cannot operate in temperatures 32° F. and below.

Examples of exemplary formulations and methods for making quat-stabilized chlorine dioxide compositions in commercial batch quantities are set forth below. Such commercial batch quantities can be made using routine methods. Smaller or larger quantities can also be made using routine methods.

Quat-Stabilized Chlorine Dioxide Teat Dips

In one exemplary embodiment, a quat-stabilized chlorine dioxide teat dip for cows or other dairy mammals is made with the following composition and using the following protocol:
Water, 82.43%
Quat-stabilized chlorine dioxide, 14.28%
CARBOPOL® Aqua SF (Lubrizol, Wickliffe, Ohio), 1.50%
Cocamidobetaine, 0.50%
Methylene Blue, 0.04%
ECOSURF™ EH-9 (Dow Chemical Company, Midland, Mich.), 1.25%

Add water to a mixing tank and begin agitation. Add CARBOPOL® Aqua SF liquid to the batch and mix for 30 minutes. Add ECOSURF™ EH-9 and cocamidobetaine. Mix for 20 minutes. Add quat-stabilized chlorine dioxide to the batch. Mix for 10 minutes. Check pH of batch. pH with caustic soda 50% to pH 7.5-8.0. Add methylene blue dye. Mix for 2 hours.

In another embodiment, a quat-stabilized chlorine dioxide teat dip for cows or other dairy mammals is made with the following composition and using the following protocol:
Water, 30.21%
Quat-stabilized chlorine dioxide, 57.12%
CARBOPOL® Aqua SF (Lubrizol, Wickliffe, Ohio), 6.00%
Cocamidobetaine, 1.80%
Methylene Blue, 0.12%
ECOSURF™ EH-9 (Dow Chemical Company, Midland, Mich.), 4.75%

Add water to a mixing tank and begin agitation. Add CARBOPOL® Aqua SF liquid to the batch and mix for 30 minutes. Add cocamidobetaine. Mix for 15 minutes. Add ECOSURF™ EH-9. Mix for 20 minutes. Add quat-stabilized chlorine dioxide to the batch. Mix for 10 minutes. Check pH of batch. pH with caustic soda 50% to pH 7.5-8.0. Add methylene blue dye. Mix for 2 hours.

Quat-Stabilized Chlorine Dioxide Navel Dip

In another exemplary embodiment, a quat-stabilized chlorine dioxide navel dip for cows or cattle is made with the following composition and using the following protocol:
Quat-stabilized chlorine dioxide, 95.46%
CARBOPOL® Aqua SF (Lubrizol, Wickliffe, Ohio), 2.50%
Methylene Blue, 0.04%
ECOSURF™ EH-9 (Dow Chemical Company, Midland, Mich.), 2.00%

Add quat-stabilized chlorine dioxide to a mixing tank and begin agitation. Add methylene blue dye to the batch and mix for 10 minutes. Add ECOSURF™ EH-9. Mix for 30 minutes. Add CARBOPOL® Aqua SF liquid to the batch. Mix for 10 minutes. pH batch with AMP-95 (pH 8.00).

Quat-Stabilized Chlorine Dioxide Hoof Spray

In another exemplary embodiment, a quat-stabilized chlorine dioxide hoof spray for cows or cattle (or other hooved mammals) is made with the following composition and using the following protocol:
Quat-stabilized chlorine dioxide, 95.00%
CARBOPOL® Aqua SF (Lubrizol, Wickliffe, Ohio), 2.50%
Pigment RED ST-2, 0.50%
ECOSURF™ EH-9 (Dow Chemical Company, Midland, Mich.), 2.00%

Add quat-stabilized chlorine dioxide to a mixing tank and begin agitation. Add ECOSURF™ EH-9 to the batch. Mix for 30 minutes. Add CARBOPOL® Aqua SF liquid to the batch. Mix for 10 minutes. pH batch with AMP-95 (pH 8.00). With agitation, add Red ST-2 pigment to the batch. Mix for 10 minutes.

Quat-Stabilized Chlorine Dioxide Dry Cow or Dairy Mammal Dip

In another embodiment, a quat-stabilized chlorine dioxide dry cow (or dairy mammal) dip is made with the following composition and using the following protocol:
Water, 16.00%
Isopropyl Alcohol, 16.00%
Dye Blue #1, 0.50%
Quat-stabilized chlorine dioxide, 2.00%
Dry Cow Resin, a proprietary blend comprising quat-stabilized chlorine dioxide available from Advanced Skin Technologies Inc. (Advanced Skin Technologies Inc., P.O. Box 579, 7143 Bernville Rd., Bernville, Pa. 19506), 65.50%.

Take half of water amount and add dye Blue #1. Pre-mix until dye is uniform. Add full amount of alcohol. Mix until uniform. Set aside. Take other half of water and add the quat-stabilized chlorine dioxide. Mix until clear. Set aside. Add clear Dry Cow Resin to mixing vessel. Start agitation. Add the quat-stabilized chlorine dioxide solution very slowly. Mix for 10 minutes until uniform. Add dye solution. Mix for 30 minutes.

Quat-Stabilized Chlorine Dioxide Gentle Cleanser

In one exemplary embodiment, a quat-stabilized chlorine dioxide gentle cleanser was made with the following composition and using the following procedure. Such a composition can be used, for example, in a gentle cleanser for skin (e.g., mammalian skin such as human skin) or can be used as a component of an exfoliating cleanser, along with beads, abrasive particles, or other abrasives or exfoliants known in the art.

Sodium methyl 2-sulfolaurate (also known as disodium 2-sulfolaurate or sodium lauryl sulfoacetate), 47.00%

Deionized water, 25.80%

Cocamidopropyl betaine, 9.60%

PRP® FG beeswax (also known as Bioboom PRP®, Universal Remediation, Inc., 450 Butler Street, Pittsburgh Pa. 15223), 5.00%. PRP® FG beeswax consists of microscopic spheres of treated, oleophilic and hydrophobic beeswax.

Quat-stabilized chlorine dioxide, 2.00%

Glycerin, 1.5%

SPECTRAGUARD™ antiscalant/dispersant (a proprietary blend of caprylyl glycol, hexylene glycol and methylisothiazolinone; Professional Water Technologies, Vista, Calif.), 0.80%

CARBOPOL®Aqua SL (Lubrizol, Wickliffe, Ohio), 8.00%

Raw honey fragrance, 0.30%

Procedure:

1. Add the deionized water to the final mixing vessel, begin propeller agitation.

2. Add CARBOPOL® liquid to the deionized water.

3. Add glycerin to the batch. Let mix for 10 minutes.

4. Add sodium methyl 2-sulfolaurate to the batch. Let mix into solution completely.

5. Add cocamidopropyl betaine to the batch. Mix until completely mixed in.

6. Add PRP® FG beeswax to the batch. Mix slowly until smooth.

7. Add SPECTRAGUARD™ to the batch and blend well.

8. Add quat-stabilized chlorine dioxide to the batch.

9. Neutralize to 6.0-6.5 using, e.g., a dispersant such as 2-amino-2-methyl-1-propanol containing 5% water (dispersant for kaolin clay, titanium dioxide, or calcium carbonate, trade name AMP-95®, ANGUS Chemical Company, Buffalo Grove, Ill.).

10. Add fragrance slowly until incorporated. Let mix slowly until smooth but thick. pH should be at 6.0-6.5. Leave as is.

Quat-Stabilized Chlorine Dioxide Hand Sanitizer

The following composition was made using the following methods. Such a composition can be used, for example, in a hand sanitizer.

Deionized water, 91.09%

Quat-stabilized chlorine dioxide, 2.90%

Pigment Blue ST 15:3 liquid (also known as Pigment Blue 62, C.I. Pigment Blue 62, C.I.42595:4, triarylmethane), 0.01%

CARBOPOL® Aqua SF (Lubrizol, Wickliffe, Ohio), 1.00%

Procedure
a) Charge vessel with deionized water.
b) While mixing, add Quat-stabilized chlorine dioxide and mix for fifteen (15) minutes.
c) Add Pigment and CARBOPOL® Aqua SF-1.
d) Mix for 20 minutes.
e) Adjust pH to 8-9 range.

Quat-Stabilized Chlorine Dioxide Hand Sanitizer

The following composition was made using methods described below. Such a composition can be used, for example, as a hand sanitizer.

Phase A:
Deionized water, 91.7440%
CARBOPOL® ULTREZ-10 (Lubrizol, Wickliffe, Ohio), 0.2000%
Sodium Hydroxide NaOH, 0.4000%

Phase B:
Glycerin 99.7% USP, 2.0000%
Dow Corning® ST -Cyclomethicone 5- NF, 1.0000%
Aloe Vera Gel, 1.0000%
Vitamin E USP, 0.1000%
Tetraethylammonium (TEA) 99%, 0.7000%

Phase C:
Quat-stabilized chlorine dioxide, 2.8560%

Procedure:

1) In the main tank, weigh Phase A water. While mixing with high agitation, slowly add ULTREZ-10 into the tank by sifting. Mix for 15 minutes or until dissolved.

2) Add Sodium Hydroxide (10% Solution) into the batch. While mixing with high agitation, create a clear and thick gel. Continue mixing for 5 minutes.

3) Add Phase B Ingredients into the batch, one at a time and mix for 5-10 minutes.

4) With high agitation, add Phase C quat-stabilized chlorine dioxide concentrate into the batch and continue mixing for 5-10 minutes.

5) Take out a sample and check pH of the batch. Adjust the pH to the range of 7.5-8.0 with TEA 99% if needed.

Benefits of Use of Compositions Comprising Quat-Stabilized Chlorine Dioxide

Non-toxic at potable water concentrations (i.e., ˜1 mg/L). Apart from its use in pulp bleaching, CIO₂ is used, at low residuals, for drinking water disinfection by many municipalities.

Rapid disinfection kinetics. CIO₂ has been found to be second only to ozone in “clean” systems and unmatched in contaminated systems. Thus those species that react slowly with CIO₂do not typically interfere with disinfection kinetics, which occurs more rapidly than the reaction of CIO₂ with many species.

Effective in highly contaminated systems. CIO₂ is unmatched as a disinfectant in systems contaminated with hydrocarbon because it reacts very slowly, if at all, with many organics.

Excellent for dissolution of biofilm. In a number of applications, CIO₂ has been found to be very effective at removal, prevention and control of biofilm, especially anaerobic biofilm. CIO₂ is polar, with a polarity that falls somewhere between that of hexane and that of water. Consequently, CIO₂ rapidly distributes into the organic phase, unlike ionic or charged species (such as hypochlorite ion), or polar molecules, (such as water). Effective at very low residuals.

CIO₂ has been found to be effective at eliminating bacteria at applied dosages that are well below the demand.

Slow bacterial recovery. Many non-oxidizing biocides used for bacterial control are poisons which may only injure bacteria, so that they can recover. CIO₂ provides a complete kill. Bacteria have not been found that are able to develop resistance to inactivation by CIO₂.

Disinfection effective over a wide range of pH. CIO₂ has been shown to be effective over a range of pH from about 4 to about 10.

Environmentally friendly. CIO₂, unlike chlorine, does not react with organics to produce carcinogenic trihalomethanes (THMs). This fact, coupled with the fact that, ultimately, CIO₂ breaks down to chloride ion, makes it the only true “green” biocide in use today.

The following examples are offered by way of illustration and not by way of limitation.

### 6. EXAMPLES

### 6.1. Example 1

### In Vitro Suspension Time-Kill Analysis

Summary

This example demonstrates the disinfectant properties of an embodiment of a quat-stabilized chlorine dioxide composition disclosed herein. 10 ml of 250 ppm quat-stabilized chlorine dioxide hand sanitizer (comprising 250 ppm quat-stabilized chlorine dioxide and water; Lot: 4328) was inoculated with 0.100 ml of each test system independently. Test systems included in this study were A. baumannii ATCC 19606, B. fragilis ATCC 43858, C. albicans ATCC 10231, C. tropicalis ATCC 750, E. aerogenes ATCC 29007, E. coli ATTC 11229, E. coli ATTC 25922, E. faecalis ATTC 29212, E. faecium ATTC 51559, H. influenzae ATCC 8149, K. oxytoca ATCC 15764, K. pneumoniae ATCC 29019, M. luteus ATCC 7468, P. mirabilis ATCC 7002, P. aeruginosa ATTC 15442, P. aeruginosa ATTC 27853, S. marcescens ATTC 14756, S. aureus ATTC 6538, S. aureus ATTC 29213, S. aureus ATTC 33592, S. epidermidis ATTC 12228, S. saprophyticus ATTC 35552, S. haemolyticus ATTC 29970, S. hominis ATTC 27844, S. pneumoniae ATTC 49619, and S. pyogenes ATTC 19615. After a contact time of 60 seconds, quat-stabilized chlorine dioxide hand sanitizer (Lot: 4328) was neutralized and enumerated for surviving test systems. Quat-stabilized chlorine dioxide hand sanitizer (Lot: 4328) achieved greater than (>) 99.99% reduction (>4 log 10) against the test system B. fragilis ATTC 43858. S Quat-stabilized chlorine dioxide hand sanitizer (Lot: 4328) achieved greater than (>) 99.999% reduction (>5 log 10) against the test systems A. baumannii ATTC 19606, C. albicans ATTC 10231, C. tropicalis ATTC 750, E. coli ATTC 11229, E. coli ATTC 25922, K. oxytoca ATTC 15764, M. luteus ATTC 7468, P. mirabilis ATTC 7002, P. aeruginosa ATTC 15442, P. aeruginosa ATTC 27853, S. marcescens ATTC 14756, S. aureus ATTC 6538, S. haemolyticus ATTC 29970, S. saprophyticus ATTC 35552, S. pneumoniae ATTC 49619, and S. pyogenes ATTC 19615 after the 60 second contact time. Quat-stabilized chlorine dioxide hand sanitizer (Lot: 4328) achieved greater than (>) 99.9999% reduction (>6 log 10) against the test systems E. faecalis ATTC 29212, E. faecium ATTC 51559, K. pneumoniae ATTC 29019, S. aureus ATTC 29213, S. aureus ATTC 33592, S. epidermidis ATTC 12228, and S. hominis ATTC 27844 after the 60 second contact time. Quat-stabilized chlorine dioxide hand sanitizer (Lot: 4328) achieved greater than (>) 99.99999% reduction (>7 log 10) against the test systems E. aerogenes ATTC 29007 and H. influenzae ATTC 8149 after the 60 second contact time.

Background

Stabilized chlorine dioxide is an EPA registered disinfectant cleaner, mold killer and very effective odor eliminator. It is ecologically sound, non-irritating to the skin and non-corrosive to treated articles. It classifies as an NSF Certified surface sanitizer, kills 99.999% of bacteria, including E. coli, Salmonella and Listeria in less than 30 seconds. This substance kills bacteria by chemically altering certain amino acids and the RNA in the cell. These amino acids (RNA) are important building blocks in the proteins that help to form cell walls. When these proteins are destroyed, the cell wall ruptures and the organism dies.

Stabilized chlorine dioxide has been proven effective against methicillin-resistant Staphylococcus aureus (MRSA), Norovirus, Legionella pneumophilia, E. coli and the H1 N1 Swine flu in testing conducted by certified independent laboratories under GLP conditions. The substance does not contain the strong chemicals that are typically used in removing mold and bacteria.

The purpose of this study was to document the efficacy of an embodiment of the composition provided herein (quat-stabilized chlorine dioxide hand sanitizer against microorganisms. An In Vitro Suspension Time-Kill Test Method was used against the selected, clinically relevant, test microorganisms with a 60 second contact time using standard methods.

Materials and Methods

This study was carried out using methods known in the art (see, e.g., “ASTM, International.” Standard Guide for Assessment of Antimicrobial Activity Using a Time-Kill Procedure. 2008. ASTM E 2315-03.)

Stability Test

The stability of the quat-stabilized chlorine dioxide at 50° C. over a period of 4 weeks was assessed by periodic determination of total concentration of available chlorine dioxide, total concentration of quats and measurement of physical properties such as pH, viscosity in a representative sample taken from the stored commercial container (i.e. PETE bottle). The chlorine dioxide concentration and the amount of quats were measured by titration methods, which are well known in the art. After the completion of 4 weeks, the product stored in commercial container at 50° C. remained within the specification for chlorine dioxide content and quats content as well as pH and it was therefore concluded that quat-stabilized chlorine dioxide is stable under typical storage conditions.

Test Microorganisms

| | |
|---|---|
| Acinetobacter baumannii | ATCC 19606 |
| Bacteroides fragilis | ATCC 43858 |
| Candida albicans | ATCC 10231 |
| Candida tropicalis | ATCC 750 |
| Enterobacter aerogenes | ATCC 29007 |
| Enterococcus faecalis | ATCC 29212 |
| Enterococcus faecium | ATCC 51559 |
| Escherichia coli | ATCC 11229 |
| Escherichia coli | ATCC 25922 |
| Haemophilus influenzae | ATCC 8149 |
| Klebsiella oxytoca | ATCC 15764 |
| Klebsiella pneumoniae | ATCC 29019 |
| Micrococcus luteus | ATCC 7468 |
| Proteus mirabilis | ATCC 7002 |
| Pseudomonas aeruginosa | ATCC 15442 |
| Pseudomonas aeruginosa | ATCC 27853 |
| Serratia marcescens | ATCC 14756 |
| Staphylococcus aureus | ATCC 6538 |
| Staphylococcus aureus | ATCC 29213 |
| Staphylococcus aureus | ATCC 33592 |
| Staphylococcus epidermidis | ATCC 12228 |
| Staphylococcus haemolyticus | ATCC 29970 |
| Staphylococcus hominis | ATCC 27844 |
| Staphylococcus saprophyticus | ATCC 35552 |
| Streptococcus pneumoniae | ATCC 49619 |
| Streptococcus pyogenes | ATCC 19615 |

Preparation of the Test Culture and Test Inoculum

Preparation of the test cultures and of the test inocula were performed using standard methods known in the art. In brief, a loop of target microorganism (from the most recent working stock or daily subculture) was transferred to a test tube containing 10 mL sterile Tryptic Soy Broth (TSB), a plate of Potato Dextrose Agar, or other appropriate medium known in the art. Test culture tubes/plates were incubated at temperatures and durations known in the art to be appropriate for each microorganism.

Fungi test cultures were directly harvested from the growth agar surface in sterile PBS, and further diluted, if required, in sterile PBS to yield the appropriate test culture inoculum targeting approximately 1×10⁸ CFU/ml. Bacteria test cultures were diluted in sterile PBS to yield the appropriate test inoculum concentration targeting approximately 1×10⁸ CFU/ml. Alternatively, the test culture was used as the test inoculum if the concentration was too low to be diluted. All test inocula were vortex mixed prior to dilution or use.

Preparation of Test and Control Substances

For each microorganism, contact time evaluated, and replicate, 10.0 mL of test substance was aseptically aliquoted into 50 mL centrifuge tubes. For each microorganism tested, 10.0 mL of control substance (sterile PBS) was aseptically aliquoted into 50 ml centrifuge tubes in the same replicate number evaluated as for the test substance. Control substances were utilized to determine the starting concentration of microorganisms (Initial Numbers Control) at time zero, using standard methods known in the art.

Time Zero Determination

A 10.0 mL volume of sterile phosphate buffered saline (PBS) was inoculated with a volume (e.g., 0.010 ml) of the prepared test inoculum at a target concentration of 1.0×10⁶ CFU/ml of control substance. The inoculated control substance was vortex mixed and a serial 1:10 dilution series performed in sterile PBS. The appropriate dilutions were plated in duplicate and to the appropriate growth supporting agar.

Inoculation of the Test Substance with Test Inoculum and Neutralization Alter Exposure

For each test substance, contact time evaluated, and replicate, a volume (e.g., 0.010 ml) of test inoculum was added to the test substance of a target concentration of 1.0×10⁶ CFU/ml of test substance. A timer was started immediately after inoculation. Vortex mixing of the inoculated test substances was used to evenly disperse inoculum.

A 1.0 ml aliquot of inoculated test substance was neutralized by addition to 9.0 ml neutralizing broth of each evaluated contact time. Additional serial 1:10 dilutions were performed in sterile neutralizer broth and plated in duplicate to the appropriate growth supporting agar.

Neutralization Validation

A Neutralization Control was performed, in single replicate, for each test microorganism per test substance, wherein the same method of neutralization used in the test was replicated (i.e. 1.0 ml of test substance per 9.0 ml of neutralizer broth) and served as the neutralization test tube. The same volume of sterile PBS (control substance) as that of the test substance used above was added to the same volume of neutralizer broth as that used in the study and served as the neutralization control tube.

All tubes were vortexed to mix, then inoculated with 0.1 ml of dilute test microorganism obtained in sterile PBS so that the final concentration of test microorganism was approximately 100 CFU/ml of test or control substance. All tubes were vortexed to mix and plated in duplicate to determine the concentration of viable test microorganism (CFU/ml).

A media sterility control was performed using methods known in the art, on each day of the study and for each media type used in the study, either by incubating an agar plate of each growth supporting medium type along with enumeration plates or plating an aliquot (0.100 ml) to growth supporting agar.

A positive (growth) and purity control was performed using methods known in the art for each microorganism on the day of the study by inoculating a sterile agar plate with each test microorganism and/or performing an isolation streak on the appropriate growth supporting agar.

Incubation of Enumeration and Control Plates

All enumeration and control plates were incubated at temperatures appropriate for each test microorganism for 18-48 hours, or until growth was observed on positive control plates. Temperatures and durations were recorded.

Results and Conclusions

The following materials and procedures were used for evaluating the ability of quat-stabilized chlorine dioxide hand sanitizer to kill a broad range of clinically relevant microorganisms, when conducted in accordance with Good Laboratory Practice Standards (GLPs) stipulated by 21 CFR Part 58.

An In-Vitro Time-Kill evaluation was performed for the test substance versus a total of 26 American Type Culture Collection (ATCC) microorganism strains, as described in the Tentative Final Monograph, (Federal Register, Vol. 59:116, Jun. 17, 1994). The percent reduction and Log 10 reduction, were compared to initial numbers.

Control was determined for each challenge species following product exposures of thirty (30) seconds, and four (4) hours. All agar-plating (enumerations) were performed in duplicate, and only dilutions resulting in countable colonies (typically 1 0-300 CF U/plate) were recorded.

Quat-stabilized chlorine dioxide sanitizer achieved greater than (>) 99.99% reduction (>4 log 10) against the test system B. fragilis ATTC 43858. Quat-stabilized chlorine dioxide hand sanitizer (Lot: 4328) achieved greater than (>) 99.999% reduction (>5 log 10) against the test systems A. baumannii ATTC 19606, C. albicans ATTC 10231, C. tropicalis ATTC 750, E. coli ATTC 11229, E. coli ATTC 25922, K. oxytoca ATTC 15764, M. luteus ATTC 7468, P. mirabilis ATTC 7002, P. aeruginosa ATTC 15442, P. aeruginosa ATTC 27853, S. marcescens ATTC 14756, S. aureus ATTC 6538, S. haemolyticus ATTC 29970, S. saprophyticus ATTC 35552, S. pneumoniae ATTC 49619, and S. pyogenes ATTC 19615 after the 60 second contact time. Quat-stabilized chlorine dioxide hand sanitizer (Lot: 4328) achieved greater than (>) 99.9999% reduction (>6 log 10) against the test systems E. faecalis ATTC 29212, E. faecium ATTC 51559, K. pneumoniae ATTC 29019, S. aureus ATTC 29213, S. aureus ATTC 33592, S. epidermidis ATTC 12228, and S. hominis ATTC 27844 after the 60 second contact time. Quat-stabilized chlorine dioxide hand sanitizer (Lot: 4328) achieved greater than (>) 99.99999% reduction (>7 log 10) against the test systems E. aerogenes ATTC 29007 and H. influenzae ATTC 8149 after the 60 second contact time.

### 6.2. Example 2

### Minimum Inhibitory Concentration (MIC)

Summary

This example demonstrates the inhibitory properties of quat-stabilized chlorine dioxide hand sanitizer against a broad range of clinically relevant and physiologically diverse microorganisms.

Study Conclusion-in-Brief

250 ppm quat-stabilized chlorine dioxide hand sanitizer (comprising 250 ppm quat-stabilized chlorine dioxide and water) demonstrated inhibitory properties against each test microorganism at concentrations at or below 12.5% (v/v) of that submitted to the test facility.

Test Microorganisms

| | |
|---|---|
| Acinetobacter baumannii | ATCC 19606 |
| Bacteroides fragilis | ATCC 43858 |
| Candida albicans | ATCC 10231 |
| Candida tropicalis | ATCC 750 |
| Enterobacter aerogenes | ATCC 29007 |
| Enterococcus faecalis | ATCC 29212 |
| Enterococcus faecium | ATCC 51559 |
| Escherichia coli | ATCC 11229 |
| Escherichia coli | ATCC 25922 |
| Haemophilus influenzae | ATCC 8149 |
| Klebsiella oxytoca | ATCC 15764 |
| Klebsiella pneumoniae | ATCC 29019 |
| Micrococcus luteus | ATCC 7468 |
| Proteus mirabilis | ATCC 7002 |
| Pseudomonas aeruginosa | ATCC 15442 |
| Pseudomonas aeruginosa | ATCC 27853 |
| Serratia marcescens | ATCC 14756 |
| Staphylococcus aureus | ATCC 6538 |
| Staphylococcus aureus | ATCC 29213 |
| Staphylococcus epidermidis | ATCC 12228 |
| Staphylococcus haemolyticus | ATCC 29970 |
| Staphylococcus hominis | ATCC 27844 |
| Staphylococcus saprophyticus | ATCC 35552 |
| Streptococcus pneumoniae | ATCC 49619 |
| Streptococcus pyogenes | ATCC 19615 |

Introduction

This example describes the materials and procedure for determining the concentration of quat-stabilized chlorine dioxide hand sanitizer that inhibits the growth when tested against a broad spectrum of clinically relevant microorganisms when conducted in accordance with Good Laboratory Practice Standards (GLPs) stipulated by 21 CFR Part 58.

The Minimum Inhibitory Concentration (MIC) was determined versus a total of 25 American Type Culture Collection (ATCC) microorganism strains, as described in the Tentative Final Monograph (Federal Register, Vol. 59:116, Jun. 17, 1994). The challenge species was exposed to each of a maximum of fifteen doubling dilutions of the product, prepared in appropriate broth and incubated for 18-24 hours (or other art-known time periods as necessary for particular test microorganisms), at which time the MIC was visually determined and recorded.

Purpose

The purpose of this example was to document the efficacy of test substance against the test systems (microorganisms) and under the test parameters specified below.

Materials and Methods

Pure cultures of all test systems (microorganisms) were used for the study.

The following materials were used:

Sufficient volume of each test substance. Test substance was 250 ppm quat-stabilized chlorine dioxide hand sanitizer (comprising 250 ppm quat-stabilized chlorine dioxide and water).

Sufficient quantity of sterile Petri dishes.

Sufficient volume of sterile growth supporting broth and agar (recorded at the time of use).

Sufficient quantity of 96 Well Microtiter Plates

Sufficient volume of Mueller- Hinton Broth (MH B) or appropriately modified Mueller-Hinton Broth.

Sufficient quantity of sterile 50 mL, 15 mL, and 1 .5 ml centrifuge tubes.

Pipet Aid(s) or similar.

Serological pipets of appropriate size, volume, and demarcation.

Protocol for GLP Minimum Inhibitory Concentration- Protocol P1271

Micropipette(s) of appropriate volume range.

Sterile micropipette tips of appropriate size.

Sufficient Phosphate-buffered saline (PBS), or equivalent, prepared in bulk, 0.9 ml, and/or 9.0 ml volumes.

Sufficient 2× Dey/Engley (D/E) Neutralizing broth prepared in bulk, 0.9 m l, and/or 9.0 ml volumes.

Microbiological torch or other flame source.

Vortex mixer.

Inoculation loop.

Sterile syringes of appropriate volume range.

Water bath.

Spray bottle containing sufficient volume of 95% ethanol.

Biohazard receptacle.

Calibrated scale.

Satellite clock.

Incubator(s) set to temperature(s) appropriate for growth of each test microorganism.

Procedure

Preparation of the Test Culture and Test Inoculum

A loop of target microorganism (from the most recent working stock or daily subculture) was transferred to a test tube containing 10 ml sterile Tryptic Soy Broth (TSB), Potato Dextrose Agar, or other appropriate medium.

Test culture tubes/plates were incubated at temperatures and durations appropriate for each microorganism.

Fungi were directly harvested from the growth agar surface in sterile Mueller Hinton Broth, and further diluted, if required, using sterile Mueller Hinton Broth to yield the appropriate test inoculum concentration targeting 1×10⁶ CFU/ml.

Bacteria were diluted in sterile Mueller Hinton Broth to yield the appropriate test inoculum concentration targeting 1×10⁶ CFU/ml.

All test inocula were vortex mixed prior to dilution or use.

Preparation of Diluted Test Substances within the 96-well Microtiter Plates

Test substance was 250 ppm quat-stabilized chlorine dioxide hand sanitizer (comprising 250 ppm quat-stabilized chlorine dioxide and water).

Ninety-six (96) well microtiter plates were prepared by adding 0.100 ml of MHB to the appropriate wells in a quantity sufficient for triple replicate tests per test substance, per test microorganism.

In triple replicate per test substance, per test microorganism, 0.100 ml of test substance was added to the first column of wells of the prepared microtiter well plates.

The test substances were serially diluted at 1:1 concentrations, in subsequent wells within their respective rows on the microtiter plate. Standard techniques were used such that the last dilution of the test substance reached a concentration expected to be approximately two dilutions beyond the MIC of the test substance for each respective test microorganism.

Dilution of the test product was conducted in large (e.g.,100 ml) volumes and then distributed to the appropriate microtiter plate wells (0.100 ml per well) to save time and harmonize dilutions across multiple microorganisms.

Dilutions of test product were prepared by measuring and appropriate weight of test substance and conducting dilutions in a 1:1 weight/volume concentration.

Inoculation of the Test Substance with Test Inoculum

Protocol for GLP Minimum Inhibitory Concentration

A 0.100 mL volume of prepared test inoculum was added to each well containing dilute test substance, thus performing an additional 1:1 dilution of the test substance.

A positive control was performed for each replicate of test substance, per test microorganism. The positive control contained 0.100 ml of MHB and was inoculated similarly to the test substance by the addition of 0.100 ml of test culture to the well.

A negative control was performed at least once each day of study to verify the sterility of the Mueller Hinton Broth used at the time of test.

Initial Numbers Control (Time Zero Determination)

A volume was harvested from the positive control wells for each test substance and a serial dilution performed in sterile PBS to determine the starting concentration of each test microorganism.

Dilutions were plated on the appropriate growth agar for each test microorganism and were incubated alongside the microtiter plates.

A media sterility control was performed on each day of the study and for each media type used in the study either by incubating on agar plate of each growth supporting medium type along with enumeration plates or plating an aliquot (0.100 ml) to growth supporting agar.

A positive (growth) control was performed for each microorganism on the day of the study by inoculating a sterile agar plate with each test microorganism and/or performing an isolation streak on the appropriate growth supporting agar.

Incubation of Microtiter Enumeration and Control Plates

All enumeration and control plates were incubated at temperatures appropriate for each test microorganism for 18-48 hours, or until growth was observed in and on positive control wells and plates, respectively. Temperatures and durations were recorded.

Observation of MIC

After the incubation period, microtiter plates were removed from the incubator and wells were observed for each test substance against each test microorganism for turbidity or pelleting indicating proliferation of the microorganism.

For each test substance tested against each test microorganism, the last well in the dilution series that was clear of microbial growth was noted as the Minimum Inhibitory Concentration of that test substance for the respective microorganisms.

Experimental Success Criteria

In the event of control failure, testing for the affected microorganism were repeated. The Initial Numbers Control, from the positive control well, demonstrated ≧1×10⁴ CFU/mL for each test microorganism. The media sterility controls were negative for growth. The positive (growth) controls were positive for growth and demonstrated a pure culture of each test microorganism.

Results and Conclusion

A total of 25 clinically relevant test microorganisms were tested to determine the Minimum Inhibitory Concentration of quat-stabilized chlorine dioxide hand sanitizer. The quat-stabilized chlorine dioxide hand sanitizer demonstrated inhibitory properties against each test microorganism at or below 12.5% (v/v) of the original concentration, thus demonstrating antimicrobial properties against a broad range of clinically relevant and physiologically diverse microorganisms.

### 6.3. Example 3

### Quat-Stabilized Chlorine Dioxide Composition as Dairy Teat Dip

In this example, an embodiment of the quat-stabilized chlorine dioxide composition (SFP—VO—2), was tested for its effectiveness for use as a disinfectant in teat cleaning in dairy cattle. The results were compared with a hydrogen peroxide-based disinfectant (SFP—OXY—TD), and 5000 ppm iodine tincture. Hydrogen peroxide-based disinfectants are well known in the dairy industry, and have been tested and used as disinfectants. Iodine tinctures are also well known as disinfectants. This example demonstrates that a quat-stabilized chlorine dioxide composition has disinfectant activity and effects that are comparable to those of a hydrogen peroxide-based disinfectant, that the quat-stabilized chlorine dioxide composition effects a complete kill, and that the effects can last for at least 8 hours, making the quat-stabilized chlorine dioxide composition extremely suitable for potential use as a teat dip in the dairy industry.

Materials and Methods

SFP—V—2 (quat-stabilized chlorine dioxide composition) had the following composition:

| | |
|---|---|
| Water | 93.88% |
| Quat-stabilized chlorine dioxide | 4.10% |
| CARBOPOL ® Aqua SF | 1.00% |
| Cocamidobetaine | 0.25% |
| Methylene Blue | 0.02% |
| ECOSURF ™ EH-9 | 0.75% |
| Total | 100.00% |

To prepare the SFP—VO—2, water was added to a tank. Agitation was begun, the liquid CARBOPOL® Aqua SF was added to the batch, and the batch was mixed for 30 minutes. ECOSURF™ EH-9 surfactant and cocamidobetaine was added to the batch and the batch was mixed for 20 minutes. Quat-stabilized chlorine dioxide was added to the batch and the batch was mixed for 10 minutes. pH of the batch was checked, and the batch was adjusted to pH 8.8 with caustic soda 50%. Methylene blue dye was added to the batch and the batch was mixed for 2 hours. A QC (quality control) sample was pulled according to standard methods.

SFP—OXY—TD had the following composition:

| | |
|---|---|
| Water | 93.35% |
| Yellow #5 | 0.01-0.04% (e.g., 47.2 g |
| | per 819.7 gal of water) |
| Red Dye #40 | 0.01-0.04% (e.g., 47.2 g |
| | per 819.7 gal of water) |
| Hydrogen peroxide | 2.05% |
| Cocoamidopropyl betaine (CALTAINE ® C) | 0.50% |
| Peroxide chelator (e.g., Versenate) | 0.10% |
| Glycerine | 4.00% |
| Total | 100.00% |

To prepare the SFP—OXY—TD, water was added to a tank and agitation was begun. Yellow #5 was added to the batch and the batch was mixed for 5 minutes. Red #40 was added to the batch and the batch was mixed for 5 minutes. Versenate (edetate calcium disodium, a peroxide chelator) was added to the batch and the batch was mixed for 10 minutes. CALTAINE® C was added to the batch and batch was mixed for 10 minutes. Glycerine was added to the batch and batch was mixed for 10 minutes. Hydrogen peroxide was added to the batch and the batch was mixed for 10 minutes. pH of the batch was checked, and the batch was adjusted to pH 5.0 -5.5 using citric acid according to standard methods A QC (quality control) sample was pulled according to standard methods.

The experiment was conducted according to the following procedures. First, iodine tincture (5000 ppm), SFP—OXY—TD and SFP—VO—2 disinfectants were sprayed on a clean experiment table on three spots at one hour intervals, for eight times consecutively and the results were compared. Table I shows numbers of colonies growing on the experiment table before and after the disinfectants were sprayed.

TABLE I
Numbers of colonies growing on experiment table before and after
disinfectants were sprayed

| | Disinfectants | | | | | | | | |
|---|---|---|---|---|---|---|---|---|---|
| | | | | | | | 5000 ppm iodine tincture | | |
| | SFP-OXY-TD | | | SFP-VO-2 | | | disinfectant | | |
| | Spraying spots | | | | | | | | |
| | 1 | 2 | 3 | 1 | 2 | 3 | 1 | 2 | 3 |
| Before the | 1.01 × 10⁶ | 2.14 × 10⁶ | 2.06 × 10⁶ | 2.24 × 10⁶ | 1.86 × 10⁶ | 1.75 × 10⁶ | 2.09 × 10⁶ | 1.13 × 10⁶ | 2.75 × 10⁶ |
| disinfectants | | | | | | | | | |
| were sprayed | | | | | | | | | |
| One hour after | 3.35 × 10⁴ | 4.46 × 10⁵ | 4.95 × 10⁴ | 1.10 × 10⁴ | 2.86 × 10⁴ | 3.24 × 10⁴ | 1.15 × 10⁴ | 1.09 × 10⁵ | 2.34 × 10⁵ |
| the | | | | | | | | | |
| disinfectants | | | | | | | | | |
| were sprayed | | | | | | | | | |
| 2 h after | 3.65 × 10⁴ | 3.36 × 10⁵ | 5.10 × 10⁴ | 4.30 × 10⁴ | 1.13 × 10⁵ | 1.29 × 10⁴ | 1.12 × 10⁴ | 1.12 × 10⁵ | 2.21 × 10⁵ |
| 3 h after | 2.85 × 10⁴ | 3.41 × 10⁵ | 3.80 × 10⁴ | 3.60 × 10⁴ | 1.05 × 10⁴ | 1.01 × 10⁴ | 1.09 × 10⁴ | 2.23 × 10⁴ | 2.01 × 10⁵ |
| 4 h after | 1.05 × 10⁴ | 5.35 × 10⁴ | 3.05 × 10⁴ | 0.55 × 10⁴ | 1.30 × 10⁴ | 1.25 × 10⁴ | 1.11 × 10⁴ | 2.13 × 10⁴ | 1.19 × 10⁴ |
| 5 h after | 0.40 × 10⁴ | 5.12 × 10⁵ | 1.61 × 10⁵ | 0.55 × 10⁴ | 1.19 × 10⁴ | 2.80 × 10⁴ | 1.14 × 10⁴ | 2.26 × 10⁴ | 2.00 × 10⁴ |
| 6 h after | 2.40 × 10⁴ | 1.92 × 10⁵ | 1.05 × 10⁶ | 1.10 × 10⁴ | 2.05 × 10⁴ | 3.15 × 10⁴ | 1.21 × 10⁴ | 2.34 × 10⁴ | 2.59 × 10⁴ |
| 7 h after | 1.20 × 10⁴ | 2.88 × 10⁵ | 1.10 × 10⁵ | 1.30 × 10⁴ | 2.50 × 10⁴ | 2.65 × 10⁴ | 1.10 × 10⁴ | 2.54 × 10⁴ | 2.76 × 10⁴ |
| 8 h after | 0.85 × 10⁴ | 9.82 × 10⁵ | 2.98 × 10⁵ | 1.15 × 10⁴ | 2.65 × 10⁴ | 2.50 × 10⁴ | 1.12 × 10⁴ | 2.34 × 10⁴ | 2.45 × 10⁴ |

In a second experiment, iodine tincture, SFP—OXY—TD and SFP—VO—2 disinfectants were sprayed on the clean experiment table on one spot at one hour intervals for eight times consecutively to determine the efficacy of these three disinfectants and to determine the duration of the disinfecting effects of these three disinfectants.

Table II shows the continued and persistent result of disinfectant sprayed onto the clean experiment table.

TABLE II
Numbers of colonies observed on clean experiment table

| | | | 5000 ppm iodine |
|---|---|---|---|
| | SFP-OXY-TD | SFP-VO-2 | tincture disinfectant |
| before the | 880 | 1016 | 788 |
| disinfectants | | | |
| were used | | | |
| one hour after | 3 | 1 | 1 |
| the disinfectants | | | |
| were used | | | |
| After 2 h | 1 | 0 | 1 |
| After 3 h | 1 | 1 | 0 |
| After 4 h | 1 | 1 | 1 |
| After 5 h | 1 | 1 | 1 |
| After 6 h | 1 | 1 | 2 |
| After 7 h | 1 | 1 | 0 |
| After 8 h | 2 | 1 | 1 |

Thirdly, the three disinfectants, i.e., iodine tincture, SFP—OXY—TD and SFP—VO—2, were mixed with milk samples containing mastitis. Germiculture was conducted on blood plates and MacConkey Agar plates and the growth conditions of germs were observed so as to characterize the disinfecting effect of these three disinfectants through comparisons. FIG. 2 shows pairs of photographs of test plates after germiculture, and before (left photo) and after (right photo) treatment with the test disinfectant. In each pair of photos, the left photo shows the blood plate before treatment with the test disinfectant. The right photo in each pair shows the blood plate after treatment with the test disinfectant. The results show that a quat-stabilized chlorine dioxide composition has disinfectant activity and effects that are comparable to those of the hydrogen peroxide-based disinfectant and the iodine tincture disinfectant.

### CONCLUSION

The three disinfectants, SFP—OXY—TD, SFP—VO—2 and iodine tincture disinfectant all demonstrate effective disinfecting effects that can last eight hours. The results show that a quat-stabilized chlorine dioxide composition has disinfectant activity and effects that are comparable to those of a hydrogen peroxide-based disinfectant, that the quat-stabilized chlorine dioxide composition effects a complete kill, and that the effects can last for at least 8 hours, making the quat-stabilized chlorine dioxide composition extremely suitable for potential use as a teat dip in the dairy industry.

A sample of the methods and compositions that are described herein are set forth in the following numbered paragraphs:

1. A method for treating skin comprising applying to the skin an aqueous composition, the aqueous composition comprising quat-stabilized chlorine dioxide and water.

2. The method of paragraph 1, wherein the skin is mammalian skin.

3. The method of paragraph 2, wherein the mammalian skin is human skin.

4. The method of paragraph 1, wherein the treating is disinfecting, sanitizing, cleaning, conditioning, exfoliating, acne treatment, or skin fungus treatment.

5. The method of paragraph 1, wherein the composition comprises a dye, a surfactant or surfactant blend, a thickener, a foam booster and/or a fragrance.

6. The method of paragraph 5, wherein the surfactant or surfactant blend is a film-forming surfactant or surfactant blend.

7. The method of paragraph 5, wherein the surfactant or surfactant blend is also the thickener.

8. The method of paragraph 5, wherein the surfactant or surfactant blend comprises ECOSURF™ EH-9, sodium lauryl sulfate (SLS), sodium lauryl ether sulfate (SLES), or cocamidobetaine.

9. The method of paragraph 1, wherein the composition comprises an acrylates copolymer.

10. The method of paragraph 9, wherein the acrylates copolymer is CARBOPOL® Aqua SF-1 or SL Polymer.

11. The method of paragraph 1, wherein the composition comprises about 1-10,000 ppm of quat-stabilized chlorine dioxide.

12. The method of paragraph 5, wherein the composition comprises about 1-10,000 ppm of quat-stabilized chlorine dioxide.

13. A method for treating a teat of a mammal comprising applying to the skin an aqueous composition, the aqueous composition comprising quat-stabilized chlorine dioxide and water.

14. The method of paragraph 13, wherein the treating is disinfecting, sanitizing or conditioning.

15. The method of paragraph 13, wherein the mammal is a dairy mammal.

16. The method of paragraph 13, wherein the mammal is a cow, pig, horse, donkey, goat, sheep, buffalo, camel, alpaca, or yak.

17. The method of paragraph 13, wherein the mammal is dry or non-lactating.

18. The method of paragraph 13, wherein the composition comprises a dye, a surfactant or surfactant blend, a thickener, a foam booster and/or a fragrance.

19. The method of paragraph 18, wherein the surfactant or surfactant blend is a film-forming surfactant or surfactant blend.

20. The method of paragraph 18, wherein the surfactant or surfactant blend is also the thickener.

21. The method of paragraph 18, wherein the surfactant or surfactant blend comprises ECOSURF™ EH-9, sodium lauryl sulfate (SLS), sodium lauryl ether sulfate (SLES), or cocamidobetaine.

22. The method of paragraph 13, wherein the composition comprises an acrylates copolymer.

23. The method of paragraph 22, wherein the acrylates copolymer is CARBOPOL® Aqua SF-1 Polymer.

24. The method of paragraph 13, wherein the composition comprises about 1-10,000 ppm of quat-stabilized chlorine dioxide.

25. The method of paragraph 18, wherein the composition comprises about 1-10,000 ppm of quat-stabilized chlorine dioxide.

26. A method for treating mastitis in a mammal comprising applying to the skin an aqueous composition, the aqueous composition comprising quat-stabilized chlorine dioxide and water.

27. The method of paragraph 26, wherein the treating is disinfecting, sanitizing or conditioning.

28. The method of paragraph 26, wherein the mammal is a dairy mammal.

29. The method of paragraph 26, wherein the mammal is a cow, pig, horse, donkey, goat, sheep, buffalo, camel, alpaca, or yak.

30. The method of paragraph 26, wherein the mammal is dry or non-lactating.

31. The method of paragraph 26, wherein the composition comprises a dye, a surfactant or surfactant blend, a thickener, a foam booster and/or a fragrance.

32. The method of paragraph 31, wherein the surfactant or surfactant blend is a film-forming surfactant or surfactant blend.

33. The method of paragraph 31, wherein the surfactant or surfactant blend is also the thickener.

34. The method of paragraph 31, wherein the surfactant or surfactant blend comprises ECOSURF™ EH-9, sodium lauryl sulfate (SLS), sodium lauryl ether sulfate (SLES), or cocamidobetaine.

35. The method of paragraph 26, wherein the composition comprises an acrylates copolymer.

36. The method of paragraph 35, wherein the acrylates copolymer is CARBOPOL® Aqua SF-1 Polymer.

37. The method of paragraph 26, wherein the composition comprises about 1-10,000 ppm of quat-stabilized chlorine dioxide.

38. The method of paragraph 31, wherein the composition comprises about 1-10,000 ppm of quat-stabilized chlorine dioxide.

39. A method for treating a mammalian navel comprising applying to the skin an aqueous composition, the aqueous composition comprising quat-stabilized chlorine dioxide and water.

40. The method of paragraph 39, wherein the treating is disinfecting, sanitizing or conditioning.

41. The method of paragraph 39, wherein the mammal is a dairy mammal.

42. The method of paragraph 39, wherein the mammal is a cow, pig, horse, donkey, mule, goat, sheep, buffalo, camel, alpaca, or yak.

43. The method of paragraph 39, wherein the composition comprises a dye, a surfactant or surfactant blend, a thickener, a foam booster and/or a fragrance.

44. The method of paragraph 43, wherein the surfactant or surfactant blend is a film-forming surfactant or surfactant blend.

45. The method of paragraph 43, wherein the surfactant or surfactant blend is also the thickener.

46. The method of paragraph 43, wherein the surfactant or surfactant blend comprises ECOSURF™ EH-9, sodium lauryl sulfate (SLS), sodium lauryl ether sulfate (SLES), or cocamidobetaine.

47. The method of paragraph 39, wherein the composition comprises an acrylates copolymer.

48. The method of paragraph 47, wherein the acrylates copolymer is CARBOPOL® Aqua SF-1 or SL Polymer.

49. The method of paragraph 39, wherein the composition comprises about 1-10,000 ppm of quat-stabilized chlorine dioxide.

50. The method of paragraph 43, wherein the composition comprises about 1-10,000 ppm of quat-stabilized chlorine dioxide.

51. A method for treating a hoof of a mammal comprising applying to the skin an aqueous composition, the aqueous composition comprising quat-stabilized chlorine dioxide and water.

52. The method of paragraph 51, wherein the treating is disinfecting, sanitizing or conditioning.

53. The method of paragraph 51, wherein the mammal is a dairy mammal.

54. The method of paragraph 51, wherein the mammal is a cow, pig, horse, donkey, mule, goat, sheep, buffalo, camel, alpaca, or yak.

55. The method of paragraph 51, wherein the composition comprises a dye, a surfactant or surfactant blend, a thickener, a foam booster and/or a fragrance.

56. The method of paragraph 55, wherein the surfactant or surfactant blend is a film-forming surfactant or surfactant blend.

57. The method of paragraph 55, wherein the surfactant or surfactant blend is also the thickener.

58. The method of paragraph 55, wherein the surfactant or surfactant blend comprises ECOSURF™ EH-9, sodium lauryl sulfate (SLS), sodium lauryl ether sulfate (SLES), or cocamidobetaine.

59. The method of paragraph 51, wherein the composition comprises an acrylates copolymer.

60. The method of paragraph 59, wherein the acrylates copolymer is CARBOPOL® Aqua SF-1 or SL Polymer.

61. The method of paragraph 51, wherein the composition comprises about 1-10,000 ppm of quat-stabilized chlorine dioxide.

62. The method of paragraph 55, wherein the composition comprises about 1-10,000 ppm of quat-stabilized chlorine dioxide.

63. An aqueous composition comprising quat-stabilized chlorine dioxide and water.

64. The composition of paragraph 63, comprising a dye, a surfactant or surfactant blend, a thickener, a foam booster and/or a fragrance.

65. The composition of paragraph 64, wherein the surfactant or surfactant blend is a film-forming surfactant or surfactant blend.

66. The composition of paragraph 64, wherein the surfactant or surfactant blend is also the thickener.

67. The composition of paragraph 64, wherein the surfactant or surfactant blend comprises ECOSURF™ EH-9, sodium lauryl sulfate (SLS), sodium lauryl ether sulfate (SLES), or cocamidobetaine.

68. The composition of paragraph 63, comprising an acrylates copolymer.

69. The composition of paragraph 68, wherein the acrylates copolymer is CARBOPOL® Aqua SF-1 or SL Polymer.

70. The composition of paragraph 64, comprising an acrylates copolymer.

71. The composition of paragraph 70, wherein the acrylates copolymer is CARBOPOL® Aqua SF-1 or SL Polymer.

72. The composition of paragraph 63 comprising about 1-10,000 ppm of quat-stabilized chlorine dioxide.

73. The composition of paragraph 64 comprising about 1-10,000 ppm of quat-stabilized chlorine dioxide.

The present invention is not to be limited in scope by the specific embodiments described herein. Indeed, various modifications of the embodiments, in addition to those described herein, will become apparent to those skilled in the art from the foregoing description. Such modifications are intended to fall within the scope of the appended claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A method for treating skin comprising applying to the skin an aqueous composition, the aqueous composition comprising:
quat-stabilized chlorine dioxide;
a film-forming surfactant or surfactant blend;
an acrylates copolymer;
a foam booster; and
water.

2. The method of claim 1, wherein the skin is mammalian skin.

3. The method of claim 2, wherein the mammalian skin is human skin.

4. The method of claim 1, wherein the treating is disinfecting, sanitizing, cleaning, conditioning, exfoliating, acne treatment, or skin fungus treatment.

5. The method of claim 1, wherein the composition comprises a dye and/or a fragrance.

6-7. (canceled)

8. The method of claim 1, wherein the film-forming surfactant or surfactant blend comprises ECOSURF™ EH-9, sodium lauryl sulfate (SLS), sodium lauryl ether sulfate (SLES), or cocamidobetaine.

9. (canceled)

10. The method of claim 1, wherein the acrylates copolymer is CARBOPOL® Aqua SF-1 or SL Polymer.

11. The method of claim 1, wherein the composition comprises about 1-10,000 ppm of quat-stabilized chlorine dioxide.

12-62. (canceled)

63. An aqueous composition comprising:
quat-stabilized chlorine dioxide;
a film-forming surfactant or surfactant blend;
an acrylates copolymer;
a foam booster; and
water.

64. The composition of claim 63, comprising a dye and/or a fragrance.

65 -66. (canceled)

67. The composition of claim 63, wherein the film-forming surfactant or surfactant blend comprises ECOSURF™ EH-9, sodium lauryl sulfate (SLS), sodium lauryl ether sulfate (SLES), or cocamidobetaine.

68. (canceled)

69. The composition of claim 63, wherein the acrylates copolymer is CARBOPOL® Aqua SF-1 or SL Polymer.

70-71. (canceled)

72. The composition of claim 63 comprising about 1-10,000 ppm of quat-stabilized chlorine dioxide.

73. (canceled)
